(19) United States
(12) Patent Application Publication
(10) Pub. No.: US 2013/0030395 A1
(43) Pub. Date: Jan. 31, 2013

(54) Connecting device for use in vacuum therapy of wounds

(71) Applicants: Pierre Croizat, Herbrechtingen (DE); Axel Eckstein, Heidenheim (DE); Cornelia Wolf, Herbrechtingen (DE)

(72) Inventors: Pierre Croizat, Herbrechtingen (DE); Axel Eckstein, Heidenheim (DE); Cornelia Wolf, Herbrechtingen (DE)

(21) Appl. No.: 13/555,209

(22) Filed: Jul. 23, 2012

Related U.S. Application Data
(60) Provisional application No. 61/513,697, filed on Aug. 1, 2011.

(30) Foreign Application Priority Data
Jul. 26, 2011 (DE) 10 2011 108 726.9

(51) Int. Cl. A61M 1/00

(52) U.S. Cl. 604/319

(57) ABSTRACT

A connecting device (2) for use in the treatment of wounds has a conduit (10) which can be evacuated and a flat, extended vacuum-tight carrier (12) on which the conduit (10) is held in a vacuum-tight fashion, wherein the carrier (12) can be disposed onto a vacuum dressing (6) covering the wound. The conduit (10) communicates with the wound space through openings. An adhesion-promoting layer (24) is provided on the side (22) of the carrier (12) facing the wound and has a middle supporting layer (26), a first adhesive layer (28) held on the supporting layer (26) and facing the carrier (12) and a second adhesive layer (30) held on the supporting layer (26) and facing away from the carrier (12). The adhesion-promoting layer does not block an opening (18) in the carrier (12).

This application claims Paris convention priority of DE 10 2011 108 726.9 file on Jul. 26, 2011 as well as benefit of provisional application 61/513,697 filed on Aug. 1, 2011 the entire disclosures of which are hereby incorporated by reference.

### BACKGROUND OF THE INVENTION

The invention concerns a connecting device for use in vacuum therapy of wounds, comprising a conduit means which can be evacuated, and an extensive, i.e. plate-shaped, vacuum-tight carrier means for the conduit means, on which the conduit means is held in a vacuum-tight fashion, wherein the carrier means can be disposed onto a vacuum dressing which extends over the wound and tightly seals it from the atmosphere, such that the conduit means communicates with the wound space through at least one opening in the conduit means, in the carrier means and in the vacuum dressing.

The treatment of wounds with vacuum, in particular deep wounds the healing process of which is a priori problematic, has recently become more and more important. Vacuum treatment thereby means that an area of a body or wound, which is, in principle, exposed to the surrounding atmosphere, is sealed from the surroundings, i.e. the atmosphere in which we live and breathe, in a pressure-tight or vacuum-tight fashion through means which are described in detail below, wherein a lower pressure than atmospheric pressure, consequently a vacuum compared to atmosphere, can be created and permanently maintained within the sealed wound area in a fashion which is also explained below. The vacuum mentioned in this connection means a pressure range which is typically between 0 and 250 mm Hg (mm mercury column) below the ambient atmospheric pressure. This has proven to support wound healing. A vacuum dressing is provided for vacuum-tight sealing, which may comprise e.g. a pressure-tight or vacuum-tight foil layer which is typically applied to a sound body area surrounding the wound to thereby tightly seal it. For introducing and maintaining a vacuum in the wound space on the basis of a means that generates vacuum, i.e. a vacuum pump in the broadest sense, the present systems for vacuum therapy of wounds may utilize conduit means which can be evacuated and cooperate with the vacuum dressing by means of a connecting device in order to supply vacuum to or into the wound space. A connecting device of the above-mentioned type is disclosed e.g. in WO 2006/052338 A2 or in DE 10 2009 060 596 A1. An additional adhesive foil that extends over the carrier means has predominantly been used up to now for connecting the connecting device to the wound dressing which, however, has not turned out to be suitable under all conditions.

It is the underlying purpose of the present invention to create a connecting device of the above-mentioned type, the mounting and sealing of which with respect to the wound dressing can be realized at technically and economically justifiable expense and also in a user-friendly fashion.

### SUMMARY OF THE INVENTION

This object is achieved in accordance with the invention in a connecting device of the above-mentioned type in that on the side of the carrier means facing the wound, an adhesion-promoting layer is provided, which has at least three layers comprising a middle supporting layer, a first adhesive layer facing the carrier means and being held on the supporting layer, and a second adhesive layer facing away from the carrier means, i.e. facing the vacuum dressing, and being held on the supporting layer, and is designed such that it does not block the at least one opening in the carrier means.

The inventive design of the connecting device is particularly advantageous, since the adhesion-promoting layer can typically be fixed in an ideal wide extension to the flat side of the extensive carrier means facing the wound, whereas up to now, an additional adhesive foil was used to cover the carrier means or the carrier means structure, thereby generating an additional third dimension, which, due to formation of folds, aggravates correct application or adherence of the connecting device to the upper side of the wound dressing facing away from the wound during practical handling. One further advantage of the inventive connecting device results from the fact that different materials are typically used for the extensive carrier means and for the wound dressing, e.g. one is made from silicone and the other is made e.g. from polyurethane, such that the adhesive of an adhesive foil cannot be optimally adjusted to both materials. This is different in the inventive connecting device, since the first adhesive layer can be optimally selected in view of the material of the carrier means independently of the second adhesive layer and the second adhesive layer can be optimally designed in view of the material of the wound dressing independently of the first adhesive layer. In this respect, it has turned out to be advantageous to produce the first adhesive layer and the second adhesive layer from different adhesive materials having different adhesive properties such that they can be selected in accordance with the specific materials of the carrier means or vacuum dressing.

In particular, it may thereby turn out to be advantageous for the carrier means to be formed from silicone and for the first adhesive layer to comprise a silicone adhesive, at least as a main component. It may also turn out to be advantageous for the second adhesive layer to comprise an acrylate adhesive, at least as a main component. Acrylate adhesives provide excellent adhesion to polyurethane surfaces. In this case, optimum adhesion to a wound dressing having a polyurethane surface can be achieved.

In accordance with a further inventive idea, it has turned out to be advantageous for the adhesive surface of the first adhesive layer facing the carrier means and/or the adhesive surface of the second adhesive layer facing the vacuum dressing to be smaller than 45 cm², in particular smaller than 40 cm², in particular smaller than 35 cm², in particular smaller than 30 cm². It has turned out that, when the adhesive material and the adhesive counterpart, i.e. the material of the carrier means or the wound dressing, are optimally matched, the adhesive surface of the connecting device may be much smaller while achieving the same adhesive effect. The above-mentioned area values refer to the unilaterally projected area of the relevant contact side, i.e. simply to the size of the flat adhesive surface of the first or second adhesive layer.

It has also turned out that, when an optimized adhesion-promoting layer is used, the adhesive force between the connecting device and the vacuum dressing can be considerably increased. For standardized comparisons, the adhesion to a smooth and previously degreased steel plate was tested. Connecting devices of the respectively intended design are thereby extensively glued onto the smooth surface of the steel plate, wherein according to standard EN 29073-3 (ISO 9073-3) a removing device is used and a movable removing clamp of the removing device is clamped to the conduit means leading away from the carrier means, and the conduit means is then loaded with a force at a pull-off angle of approximately 0° with respect to the steel plate, i.e. in the direction of the conduit means leading away from the carrier means. The steel plate with the carrier means is thereby vertically oriented in the tensile testing machine. The steel plate is a steel plate according to DIN EN 1939. The distance between the carrier means and the clamping point is 20 cm.

Removal of the movable removing clamp is performed with a speed of 300 mm/min. In contrast to conventional connecting devices, which were either removed from the steel plate or in which the conventional adhesive foil covering the carrier means was removed from the carrier means, i.e. was destroyed, at removal forces of less than 40 N, a connecting device according to the inventive design withstands a removal force of at least 40 N without being destroyed.

It has turned out to be particularly advantageous to use an adhesion-promoting layer with the inventive connecting device, the first adhesive layer of which is designed in such a fashion that the adhesion-promoting layer has a peeling force, provided by the first adhesive layer, with respect to a steel plate of 25 to 40 N/100 mm (100 mm width of the first adhesive layer) and/or the second adhesive layer of which is designed in such a fashion that the adhesion-promoting layer has a peeling force, provided by this second adhesive layer, with respect to a steel plate of 70 to 85 N/100 mm (100 mm width of the second adhesive layer). In this connection, the adhesion-promoting layer as such is tested, i.e. not in combination with the carrier means. This peeling force is determined according to the ASTM testing method D 3330 M, wherein the dwell times are ≧1 min, the pull-off angle is 180° (peel-off orientation) and the removal speed is 300 mm/min. The test is performed at 22° C. and 50% relative air humidity. These values can advantageously be achieved when the first adhesive layer comprises a silicone adhesive or when the second adhesive layer comprises an acrylate adhesive.

With respect to production technology, it has turned out to be advantageous for the first and the second adhesive layer to have the same wide extension in the projection on their planes of extension, i.e. are congruent in the projection, and have, in particular, the same extension as the supporting layer. In the latter case, all layers are designed to be congruent. This design has turned out to be particularly advantageous since in this case, the adhesion-promoting layer can be produced on the basis of an endless flat material tape that is coated on both sides with adhesive material in that the sections required for the adhesion-promoting layer merely have to be cut to length or punched out.

In a particularly advantageous fashion, the adhesion-promoting layer can be produced on the basis of a double-sided adhesive tape, wherein a section of the double-sided adhesive tape is separated and correspondingly configured with respect to size and shape and subsequently applied to the side of the carrier means facing the wound.

The first carrier layer of the adhesion-promoting layer is advantageously applied directly onto the side of the carrier means facing the wound.

It has also turned out to be advantageous for the first and the second adhesive layers to have a thickness of 20 to 400 μm.

The middle supporting layer may advantageously be a nonwoven material, a flat material having a textile structure, such as a knitted or woven fabric, or a plastic sheet, a metal foil or a composite material thereof.

It has also turned out to be advantageous for the adhesion-promoting layer to extend over the entire side of the carrier means facing the wound, wherein in this case, the at least one opening in the carrier means is exempted for vacuum communication purposes.

It is also feasible and advantageous for the adhesion-promoting layer to extend like a frame around the at least one opening in the carrier means. In this case, the wide extension of the adhesion-promoting layer may also be considerably less than the wide extension of the side of the carrier means facing the wound. However, it has also turned out to be advantageous for the wide extension of the adhesion-promoting layer to be at least 50% of the area of the side of the carrier means facing the wound.

It has also turned out to be advantageous for the second adhesive layer to be covered by a removable protective layer, which is advantageously designed in two parts and advantageously has a handling tab and/or an area that extends past the second adhesive layer and can be grasped.

The invention further proposes to design the conduit means to be flexible and flat and be mounted with one flat side to an upper side of the carrier means facing away from the wound in order to ensure that the connecting device is convenient for the patient or causes minimum pain upon load or contact.

It has also turned out to be advantageous for the conduit means to be undetachably and extensively connected to the carrier means with at least 70% of its area projected perpendicularly onto the carrier means in a longitudinal section on the wound side for the intended use, wherein the conduit means is formed from a flexible elastomeric material, advantageously of a Shore A hardness of maximally 60. It has also turned out to be advantageous for the thickness of the composite of the conduit means and the carrier means to be maximally 7 mm.

The conduit means therefore does not have a tubular shape with a substantially round cross-section but has a flat shape, the width of which is substantially larger than the thickness thereof. In combination with the material selection, one obtains a resilient conduit means which is more pleasant for the patient when a contact pressure is applied to the connecting device or the conduit means. For this reason, point load is reduced, which is naturally painful and highly problematic, in particular, when the wound is fresh and sensitive to pain. Moreover, due to the flat design, the danger of getting hooked or caught is reduced. When the flat longitudinal section of the conduit means is connected to the flat carrier means over at least 70% of its area, projected perpendicularly onto the carrier means (wherein in this case the opening or openings in the wall of the conduit means are included), a pressure load is distributed over a larger area of the wound dressing, which is very advantageous in view of the above-mentioned problems. In the longitudinal section used for mounting, at least 80%, in particular at least 90%, and moreover, in particular at least 95% of the area of the conduit means projected perpendicularly onto the carrier means, is advantageously connected to the carrier means.

The above-mentioned width of the flat conduit means is at least 10 mm, in particular, at least 15 mm and moreover, in particular, at least 18 mm and in particular, at most 30 mm and moreover, in particular, at most 25 mm.

The elastomeric material from which the conduit means is formed, advantageously has a Shore A hardness of 5 to 60, in particular of 10 to 60, in particular of 15 to 50, in particular of 15 to 40 and moreover, in particular of 15 to 35. As mentioned above, the Shore A hardness is determined according to DIN 53505 of August 2000, i.e. at 23° C. on a plate-shaped flat and smooth sample of a thickness of 6 mm as described in the standard. According to a preferred embodiment of the invention, the conduit means is designed on the basis of silicone.

Since the flat and flexible conduit means is used to supply vacuum into the wound space and, if necessary, to supply rinsing liquids or rinsing gases and to discharge wound exudate, i.e. it preferably only has a channel-forming communication function, it is proposed not to form the conduit means in a laminated fashion with several components or layers but to form it, despite its flat design, in a tubular shape, i.e. having a cross-section extending continuously in one piece from one single material in the peripheral direction.

It is also advantageous for the conduit means to have means which are formed on the interior and are designed, in particular, in one piece with the material of the conduit means for preventing collapse of the conduit means in case of vacuum load. These means for preventing collapse of the conduit means may, in particular, be provided in a conduit means of tubular shape as described above. These means for preventing collapse may e.g. be formed by ribs or projections. In a further development of this inventive idea, they advantageously extend in a continuous fashion. The conduit means can then advantageously be formed as an extrusion element.

It may also be advantageous for the conduit means to have several channels that are separated from each other in a pressure-tight fashion, wherein, in this case, the conduit means is advantageously also designed in one piece, i.e. is not combined from several separate channel-forming means. The plurality of channels may comprise a rinsing channel that can direct a rinsing medium towards the end of the connecting device facing the wound, and a vacuum-carrying channel which is used for supplying a vacuum or discharging wound exudates. Any clogging within the conduit means may thereby also be eliminated. Each channel thereby communicates with at least one opening in the conduit means.

The advantageously flat conduit means preferably extends over a certain distance in the longitudinal direction and may then merge into a normal, more torsion-resistant round hose via a transition or coupling element (not shown) which may form a plug connection or adhesive connection. The round hose is guided to a device which generates vacuum and may be designed in the form of a stationary device or a portable device that can be carried on the body of the patient. The transition or coupling element may also be designed for coupling a multi-channel conduit means with a multi-channel round hose. A section of 10 to 60 cm has turned out to be a useful longitudinal extension of the flat conduit means.

The extensive carrier means of the connecting device, to which the advantageously flat conduit means is connected in a vacuum-tight fashion by the manufacturer, is advantageously also formed from a flexible elastomeric material of a Shore A hardness of 5 to 60, in particular of 10 to 60, in particular of 15 to 50, in particular of 15 to 40, and moreover, in particular, of 15 to 35. The extensive carrier means advantageously has a thickness of 0.75 to 3 mm, in particular of 1 to 3 mm. In accordance with a preferred embodiment of the invention, it is formed on the basis of silicone. In accordance with a further preferred embodiment, the conduit means and the carrier means are formed from the same elastomeric material. The carrier means is used to hold and uniformly support the flat conduit means in a longitudinal end section that communicates with the wound space. Its wide extension is therefore larger than the wide extension of the conduit means in the respective longitudinal end section on the wound side. In this respect, it has turned out to be advantageous for the wide extension of the carrier means to comprise at least 1.5 times, advantageously at least twice the area of the conduit means projected perpendicularly onto the carrier means, since in this case, the forces introduced via the conduit means upon contact are distributed over a larger surface and also the transfer of bending moments, applied to the conduit means, to the vacuum dressing is eliminated or reduced. They are better absorbed by the plate-shaped carrier means. It has turned out to be sufficient for the above-mentioned area ratio to be at most 5, in particular, at most 4, wherein a ratio of 2 to 3 has turned out to be advantageous.

The flat conduit means could e.g. have a rectangular cross-sectional shape, wherein the two narrow sides may also and advantageously be rounded. In accordance with a further embodiment of the invention, the conduit means has a trapezoidal cross-sectional shape. The narrow sides are inclined at an angle of inclination with respect to the plane of the flat carrier means of e.g. 25° to 60°, in particular of 35° to 50°, wherein the flanks of the unilateral or advantageously bilateral trapezoidal shape need not necessarily extend in a straight line but may also be rounded.

The vacuum-tight connection between the flat conduit means and the upper side of the carrier means facing away from the wound is of functional significance. This could basically be realized with an adhesive connection using an adhesion-promoting agent in the broadest sense. Moreover, a thermal joining connection also turned out to be advantageous, which may correspond to a type of vulcanizing connection. The previously separately produced conduit means may e.g. be applied to the instantaneously freshly poured out and only partially solidified flat carrier means such that a material-bonding intimate connection between the two components is achieved without using an additional adhesion-promoting agent.

It has turned out to be advantageous for the openings in the conduit means and in the carrier means, which communicate with one another, to overlap each other, i.e. to be aligned with each other. This can be achieved in the simplest way by simultaneously forming these openings in both components only after vacuum-tight connection of the flat conduit means and the flat carrier means. This may be achieved e.g. by a punching process extracting material.

In view of the number and the size of the openings in the conduit means and in the flat carrier means, it would per se be feasible to only provide one opening. It is, however, also advantageous to provide several openings, in particular, at least two, in particular, at least four openings per each cm of length of the conduit means in the vacuum-tight connecting area between conduit means and carrier means.

It has also turned out to be advantageous for the clear opening area of the openings to be 5 to 50% of the area of the flat sides of the conduit means and the carrier means, which are undetachably joined to each other.

Each of the above-described features are considered to be important to the invention individually and in any combination with each other and with other features. Further features, details and advantages of the invention can be extracted from the attached claims and the drawing and the following description of a preferred embodiment of the invention.

### BRIEF DESCRIPTION OF THE DRAWING

FIG. 1 shows a perspective view, which is not true to scale, of an inventive connecting device for use in connection with vacuum treatment of wounds, as well as an indicated wound with a vacuum wound dressing; and

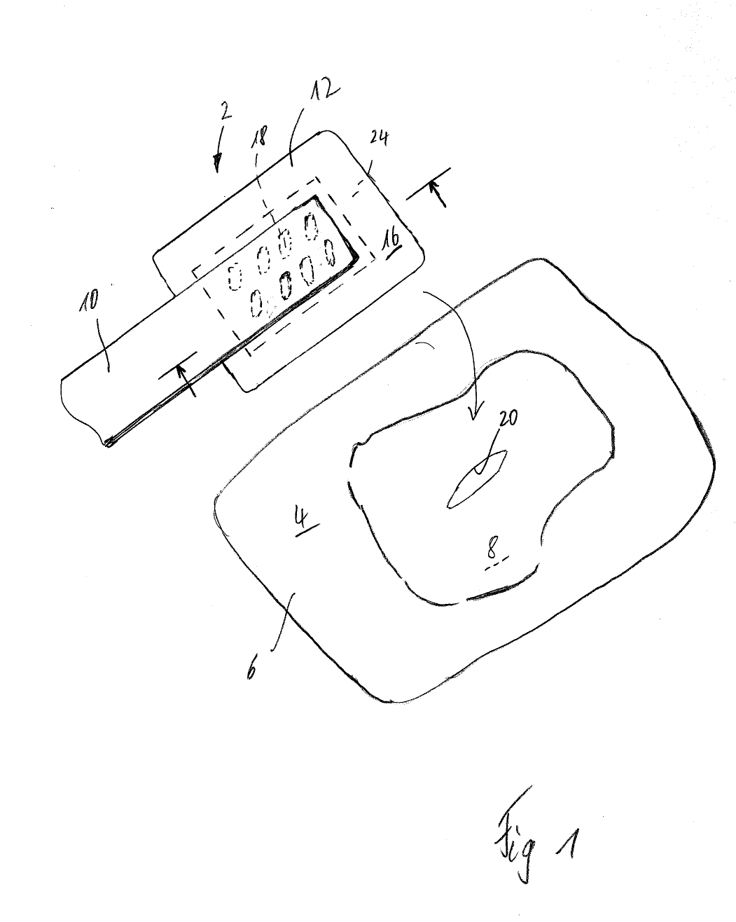

FIG. 2 shows a schematic sectional view of the connecting device according to FIG. 1, which is not true to scale.

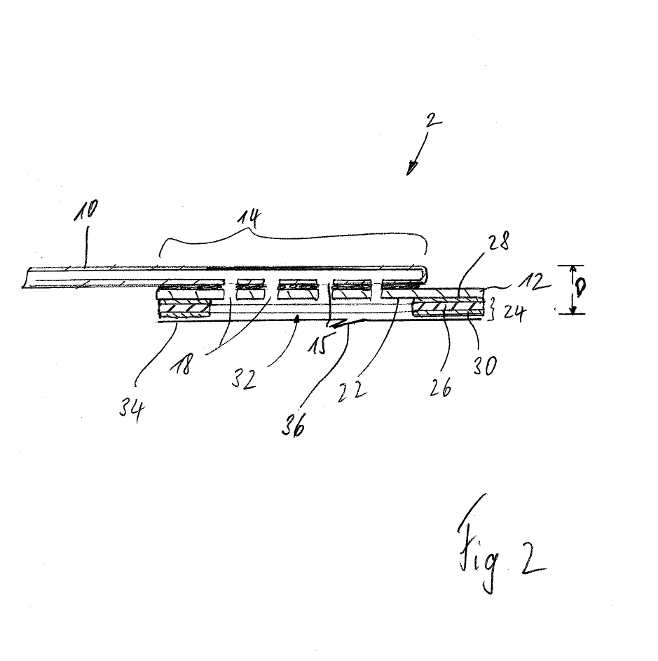

### DESCRIPTION OF THE PREFERRED EMBODIMENT

The figures show different views of an inventive connecting device, designated in total with reference numeral 2, for use in the vacuum treatment of wounds. The illustrated connecting device 2 is applied to and glued (in a fashion described below) to an upper side 4, facing away from the wound, of a schematically illustrated vacuum dressing 6 which extends over a wound 8 to be treated and is sealed in a vacuum-tight fashion with respect to atmosphere.

The connecting device 2 has a flat conduit means 10, which is shown by way of example and is made from an elastomeric flexible material, and a widely extending plate-shaped carrier means 12, which holds and supports the flat conduit means 10 such that pressure and bending forces applied onto the conduit means are uniformly introduced into a flat, extended carrier means 12 where they are absorbed.

The conduit means 10 is designed to be flat such that a longitudinal end section 14 thereof is connected to the carrier means 12 with almost 100% of its area perpendicularly projected onto the carrier means 12. The conduit means 10 may be glued or vulcanized, i.e. thermally joined, with its respective flat side 15 onto the upper side 16 of the carrier means 12 facing away from the wound.

Openings 18 that extend through the carrier means 12 and the conduit means 10 are advantageously formed after vacuum-tight joining of the conduit means 10 and the carrier means 12, the openings 18 communicating with a schematically illustrated opening 20 or several openings 20 in the vacuum dressing 6 when the connecting device 2 is applied to the vacuum dressing 6 in order to apply a vacuum to the wound space.

The thickness D of the composite of conduit means 10 and carrier means 12 is at most 7 mm, advantageously at most 5 mm and moreover advantageously only 3 to 4 mm.

The figures also show that the connecting device 2 has an adhesion-promoting layer 24 on the side 22 of the carrier means 12 facing the wound. By means of this adhesion-promoting layer 24, the connecting device 2 can be adhesively applied to the surface 4 of the vacuum dressing 6 facing away from the wound. The adhesion-promoting layer 24 has three layers and comprises a middle supporting layer 26, a first adhesive layer 28 being held on the supporting layer 26 and facing the carrier means 12, and a second adhesive layer 30 being held on the supporting layer 26 and facing away from the carrier means 12, i.e. facing the vacuum dressing 6. In the present exemplary and preferred case, all three layers of the adhesion-promoting layer 24 are designed to be congruent with each other. The adhesion-promoting layer 24 is cut to length or punched out as a whole from a flat material that is coated with adhesive material on both sides. In the exemplary case, it has the shape of a frame, i.e. is continuous in the peripheral direction and extends around that central part 32 of the carrier means 12, in which the schematically illustrated openings 18 of the carrier means 12 or of the conduit means 10 open towards the vacuum dressing 6. The adhesive surface of the first and the second adhesive layer 28, 30 is only approximately 30 cm² in the exemplary case. At this point, it should also be explicitly mentioned that the adhesive layer 24 may also extend over the entire side 22 of the carrier means 12 facing the wound, wherein in this case, it must have openings that are aligned or communicate with the openings 18 in the carrier means or conduit means. The adhesive surface is nevertheless only approximately 40 cm².

The adhesive layers 28 and 30 are formed by different adhesive materials, which are correspondingly optimized for the materials of the carrier means 12 and the vacuum dressing 6. When the carrier means 12 is e.g. made from silicone, it is advantageous for the first adhesive layer 28 to comprise a silicone adhesive. When the vacuum dressing 6, for which the connecting device 2 is designed, is formed from polyurethane or has an upper side 4 of polyurethane facing away from the wound, it is advantageous for the second adhesive layer 30 to comprise an acrylate adhesive that bonds particularly well to polyurethane. The use of the inventive adhesive layer 24 enables optimal matching of the two adhesive layers 28 and 30 thereof to the materials of the carrier means 12 and the vacuum dressing 6, which adhesively cooperate therewith, and can be designed and their composition selected by the manufacturer.

The second adhesive layer 30 is moreover covered by a two-part removable protective layer 34, which has a handling tab 36 for removing the two parts.

We claim:

1. A connecting device for use in the vacuum treatment of a wound, the wound having a wound space covered by a vacuum dressing which extends over the wound space and tightly seals it from atmosphere, the vacuum dressing having a vacuum dressing opening, the connecting device comprising:
a conduit means having at least one conduit means opening, said conduit means structured for evacuation thereof;
a flat, extended vacuum-tight carrier means on which said conduit means is held in a vacuum-tight fashion, said carrier means having a carrier means opening, said carrier means structured for cooperation with the vacuum dressing, thereby effecting communication between said conduit means and the wound space through said conduit means opening, said carrier means opening and said vacuum dressing opening; and
an adhesion-promoting layer disposed on a side of said carrier means facing the wound, said adhesion-promoting layer comprising a middle supporting layer, a first adhesive layer held on said supporting layer and facing said carrier means and a second adhesive layer held on said supporting layer and facing away from said carrier means, wherein said adhesion-promoting layer is disposed, structured and dimensioned to avoid blockage of said carrier means opening.

2. The connecting device of claim 1, wherein said first adhesive layer and said second adhesive layer are formed by different adhesive materials having different adhesive properties.

3. The connecting device of claim 1, wherein said carrier means is formed from silicone and said first adhesive layer comprises a silicone adhesive.

4. The connecting device of claim 1, wherein said second adhesive layer comprises an acrylate adhesive.

5. The connecting device of claim 1, wherein an adhesive surface of said first adhesive layer facing said carrier means and/or an adhesive surface of said second adhesive layer facing the vacuum dressing is/are smaller than 45 cm², smaller than 40 cm², smaller than 35 cm² or smaller than 30 cm².

6. The connecting device of claim 1, wherein the connecting device, when glued to a smooth steel plate by means of said second adhesive layer, withstands, at a pull-off angle of 0° with respect to the steel plate, a removal force of at least 40 N, at least 50 N, at least 60 N, at least 70 N, at least 80 N or at least 90 N without being destroyed.

7. The connecting device of claim 1, wherein said first adhesive layer is designed in such a fashion that said adhesion-promoting layer has a peeling force provided by said first adhesive layer with respect to a steel plate of 25 to 40 N/100 mm, 27 to 35 N/100 mm and/or said adhesion-promoting layer has a peeling force provided by said second adhesive layer with respect to a steel plate of 70 to 85 N/100 mm or 70 to 80 N/100 mm.

8. The connecting device of claim 1, wherein said first and said second adhesive layers have a same wide extension projected onto a plane of extension thereof.

9. The connecting device of claim 8, wherein said wide extension matches an extension of said supporting layer.

10. The connecting device of claim 1, wherein said first and said second adhesive layers have a thickness of 20 to 400 μm.

11. The connecting device of claim 1, wherein said supporting layer comprises or is formed of a non-woven material, a flat material having a textile structure, a knitted, crocheted or woven fabric or a plastic foil, a metal sheet or a composite material thereof.

12. The connecting device of claim 1, wherein said adhesion-promoting layer extends over an entire side of said carrier means facing the wound.

13. The connecting device of claim 1, wherein said adhesion-promoting layer extends like a frame around said carrier means opening.

14. The connecting device of claim 1, wherein a wide extension of said adhesion-promoting layer is at least 50% of an area of a side of said carrier means facing the wound.

15. The connecting device of claim 1, wherein said second adhesive layer is covered by a removable protective layer, which is preferably designed in two parts and advantageously comprises a handling tab and/or an area that projects past said second adhesive layer and can be grasped.

16. The connecting device of claim 1, wherein said conduit means is flexible and flat and is mounted, at a flat side thereof, to an upper side of said carrier means facing away from the wound.

17. The connecting device of claim 16, wherein said conduit means is formed from a flexible elastomeric material and is undetachably and widely connected to said carrier means for an intended use at a wound-sided longitudinal section over at least 70% of a surface thereof, projected perpendicularly onto said carrier means, wherein a composite of said conduit means and said carrier means has a thickness of at most 7 mm.

18. The connecting device of claim 17, wherein said conduit means has a Shore A hardness of at most 60.
